(19) United States
(12) Patent Application Publication
(10) Pub. No.: US 2016/0016014 A1
(43) Pub. Date: Jan. 21, 2016

(54) METHODS FOR IMPROVING BALANCE

(71) Applicant: Highland Instruments, Inc., Somerville, MA (US)

(72) Inventors: Timothy Andrew Wagner, Somerville, MA (US); Laura Dipietro, Cambridge, MA (US); Uri Tzvi Eden, Somerville, MA (US)

(21) Appl. No.: 14/800,824

(22) Filed: Jul. 16, 2015

Related U.S. Application Data
(60) Provisional application No. 62/026,291, filed on Jul. 18, 2014.

(51) Int. Cl. A61N 7/00; A61N 1/20

(52) U.S. Cl. CPC A61N 7/00; A61N 1/20; A61N 2007/0026

(57) ABSTRACT

The invention generally relates to methods for improving balance of a subject. In certain embodiments, methods of the invention involve noninvasively providing stimulation to a central nervous system of an awake subject to modulate a signal sent to or from the awake subject's central nervous system, such that the stimulation to the central nervous system improves balance of the subject.

### RELATED APPLICATION

The present application claims the benefit of and priority to U.S. provisional patent application Ser. No. 62/026,291, filed Jul. 18, 2014, the content of which is incorporated by reference herein in its entirety.

### GOVERNMENT SUPPORT

This invention was made with Government support under Grant Number 5R44NS080632 awarded by the National Institute of Neurological Disorders and Stroke (NINDS) of the National Institute of Health (NIH). The Government has certain rights in this invention.

### FIELD OF THE INVENTION

The invention generally relates to methods for improving balance of a subject.

### BACKGROUND

Parkinson's disease (PD) is a chronic and progressive movement disorder. Nearly one million people in the United States are living with Parkinson's disease. Parkinson's disease involves malfunction and death of vital nerve cells in the brain, called neurons. Parkinson's disease affects neurons in an area of the brain known as the substantia nigra. Some of those dying neurons produce dopamine, a chemical that sends messages to the part of the brain that controls movement and coordination. As Parkinson's disease progresses, the amount of dopamine produced in the brain decreases, leaving a person unable to control movement normally. Parkinson's disease can also be defined as a disconnection syndrome, in which PD-related disturbances in neural connections among subcortical and cortical structures can negatively impact the motor systems of Parkinson's disease patients and further lead to deficits in cognition, perception, and other neuropsychological aspects seen with the disease (Cronin-Golomb Neuropsychology review. 2010; 20(2):191-208. doi: 10.1007/s11065-010-9128-8. PubMed PMID: 20383586; PubMed Central PMCID: PMC2882524).

One of the most important symptoms of Parkinson's disease is postural instability, a tendency to be unstable when standing upright. Some people develop a dangerous tendency to sway when rising from a sitting position, standing, or turning. People with balance problems may have particular difficulty when pivoting or making turns or quick movements. Balance problems also lead to increased likelihood of falls. Similarly, Parkinson's disease patients also develop a problem with their ability to walk normally.

Medications have been used to reduce or eliminate symptoms of Parkinson's disease. Over time, however, the medications have reduced efficacy and show increased occurrence of side effects such as dyskinesias. Furthermore, there is strong debate whether typical medications to treat PD have any direct impact on balance instabilities.

Stimulation techniques, such as deep brain stimulation (DBS), have been used to reduce or eliminate symptoms of Parkinson's disease. However, deep brain stimulation is invasive, requiring that electrodes be implanted within the person's scalp. Furthermore, long-term effects of DBS on balance and locomotion, often referred to as axial motor signs, are debated. Additionally, DBS efficacy decreases over time as the body adjusts to stimulation and protein buildup around electrode leads attenuates the electrical field.

### SUMMARY

The invention provides methods for improving balance of a subject by noninvasively stimulating a central nervous system (e.g., brain and/or spinal cord) of the subject. Since methods of the invention use noninvasive stimulation, the stimulation is easily tuned over time, maintaining the efficacy of the treatment. Aspects of the invention are accomplished by noninvasively providing stimulation to a central nervous system of an awake subject to modulate a signal sent to or from the awake subject's central nervous system. The stimulation to the central nervous system improves balance of the subject. Generally, the signal will be processed in the subject's brain. However, the signal may be processed in other parts of the subject's body, e.g., the spinal cord. In certain embodiments, effects of the stimulation alter neural function past the duration of stimulation. Thus, the effects of the treatment last significantly longer than the period of treatment.

The parameters of the stimulation can be tuned so that stimulation is provided in a short period of time, allowing for a subject to receive stimulation while awake with little disruption to their day. That is opposed to stimulation protocols that require that stimulation be provided for long periods of time while a subject sleeps. In certain embodiments, the stimulation is provided in a single session that lasts 3 hours or less. For example, a single session may last 2.5 hours or less, 2 hours or less, 1 hour or less, thirty minutes or less, 25 minutes or less, 20 minutes or less, 15 minutes or less, 10 minutes or less, or 5 minutes or less. An exemplary stimulation protocol may involve numerous stimulation sessions over multiple days, with no single session lasting more than three hours. For example, a stimulation protocol may be two weeks in length, in which a subject received a single 20 minute session of stimulation each day of the week, or 5 days/week over a two week period (e.g., on weekdays). The stimulation can be tuned such that nothing more than stimulation of the central nervous system is required to improve the subject's balance, i.e., the methods of the invention are performed without additionally stimulating the peripheral nervous system and/or without the use of a therapeutic agent. In other embodiments, the stimulation is provided in combination with use of a therapeutic agent. In those embodiments, the stimulation enhances the efficacy of the medication and the medication and the stimulation work in combination to improve the subject's balance. In other embodiments in which stimulation is provided in conjunction with a therapeutic agent, the therapeutic agent can be used to affect one part of a disease and stimulation another part of the disease, but the different effects could be used together to improve a patient's balance.

Typically, the subject is afflicted with a movement disorder. Exemplary movement disorders include Parkinson's disease, Parkinsonism (aka., Parkinsonianism which includes Parkinson's Plus disorders such as Progressive Supranuclear Palsy, Multiple Systems Atrophy, and/or Corticobasal syndrome and/or Cortical-basal ganglionic degeneration), tauopathies, synucleinopathies, Dementia with Lewy bodies, Dystonia, Cerebral Palsy, Bradykinesia, Chorea, Huntington's Disease, Ataxia, Tremor, Essential Tremor, Myoclonus, tics, Tourette Syndrome, Restless Leg Syndrome, and/or Stiff Person Syndrome.

Any type of noninvasive stimulation known in the art may be used with methods of the invention, and the stimulation may be provided in any clinically acceptable manner. Exemplary types of stimulation include mechanical, optical, electromagnetic, thermal, or a combination thereof. In particular embodiments, the stimulation is a mechanical field (i.e., acoustic field), such as that produced by an ultrasound device. In other embodiments, the stimulation is an electrical field. In other embodiments, the stimulation is a magnetic field. Other exemplary types of stimulation include Transcranial Direct Current Stimulation (TDCS), Transcranial Ultrasound (TUS)/Transcranial Doppler Ultrasound (TDUS), Transcranial Electrical Stimulation (TES), Transcranial Alternating Current Stimulation (TACS), Cranial Electrical Stimulation (CES), or Transcranial Magnetic Stimulation (TMS). In other embodiments, the stimulation source may work in part through the alteration of the nervous tissue electromagnetic properties, where stimulation occurs from an electric source capable of generating an electric field across a region of tissue and a means for altering the permittivity of tissue relative to the electric field, whereby the alteration of the tissue permittivity relative to the electric field generates a displacement current in the tissue. The means for altering the permittivity may include a chemical source, optical source, mechanical source, thermal source, or electromagnetic source. It is known that the skull bone is permeable to chemical agents and that chemical agents can be applied transcranially to brain tissue, such as described for example in Pathirana et al. (Indian J Pharm Sci, 68:493-496, 2006), the content of which is incorporated by reference herein in its entirety. Similarly, it is known that optical energy can be delivered transcranially, such as described for example in DeTaboada et al. (Lasers in Surgery and Medicine, 38(1):70-73, 2006), the content of which is incorporated by reference herein in its entirety.

In other embodiments, the stimulation is provided by a combination of an electric field and a mechanical field. The electric field may be pulsed, time varying, pulsed a plurality of time with each pulse being for a different length of time, or time invariant. Generally, the electric source is current that has a frequency from about DC to approximately 100,000 Hz. The mechanical field may be pulsed, time varying, or pulsed a plurality of time with each pulse being for a different length of time. In certain embodiments, the electric field is a DC electric field.

The stimulation may be applied to a structure or multiple structures within the brain or the nervous system. Exemplary structures include dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, specific tracts of the spinal cord, and spinal cord.

In one exemplary embodiment, the electric field is applied broadly and mechanical field is focused on a specific brain structure or multiple structures for therapeutic purposes. The electric field may be applied broadly and the mechanical field may be focused on a structure or multiple structures, such as brain or nervous tissues including dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, cortical structures, sub-cortical structures, and/or spinal cord. Other possible configurations include applying both the electrical field and the mechanical field in a broad manner; applying both the electric field and the mechanical field in a focused manner; or applying the electric field in a focused manner and the mechanical field in a broad manner.

### BRIEF DESCRIPTION OF THE DRAWINGS

FIG. 1 is a plan view of one embodiment of an apparatus for stimulating biological tissue constructed in accordance with the principles of the present disclosure.

FIG. 2 is a top plan view of an exemplary embodiment of an apparatus for stimulating biological tissue constructed in accordance with the principles of the present disclosure.

FIG. 3 is a top plan view of an exemplary embodiment of an apparatus for stimulating biological tissue implementing a chemical source for altering permittivity constructed in accordance with the principles of the present disclosure.

FIG. 4 is a top plan view of an exemplary embodiment of an apparatus for stimulating biological tissue implementing a radiation source for altering permittivity constructed in accordance with the principles of the present disclosure.

FIG. 5 is a top plan view of another exemplary embodiment of an apparatus for stimulating biological tissue implementing an optical beam for altering permittivity constructed in accordance with the principles of the present disclosure.

FIG. 6 is a graph showing that patients that received Electrosonic stimulation as compared to SHAM had improved balance with eyes open.

FIG. 7 is a graph showing that patients that received Electrosonic stimulation as compared to SHAM had improved balance with eyes closed.

FIG. 8 is an illustration showing stimulation improves motor function, such as balance.

FIG. 9 is a graph showing GAIT improvement.

FIG. 10 is a graph showing Walking Time Improvement.

### DETAILED DESCRIPTION

Parkinson's disease occurs in part as the result of insufficient quantities of the neurotransmitter dopamine in a part of the brain called the substantia nigra. The substantia nigra helps in the planning and control of movement. Dopamine levels are reduced as the neurons that produce dopamine die. As a result, messages concerning the planning and control of movement are interrupted. Additionally, PD-related disturbances in neural connections among subcortical and cortical structures can negatively impact the motor systems of PD patients, and further lead to deficits in cognition, perception, and other neuropsychological aspects seen with the disease. One result of such function losses is impaired balance and coordination.

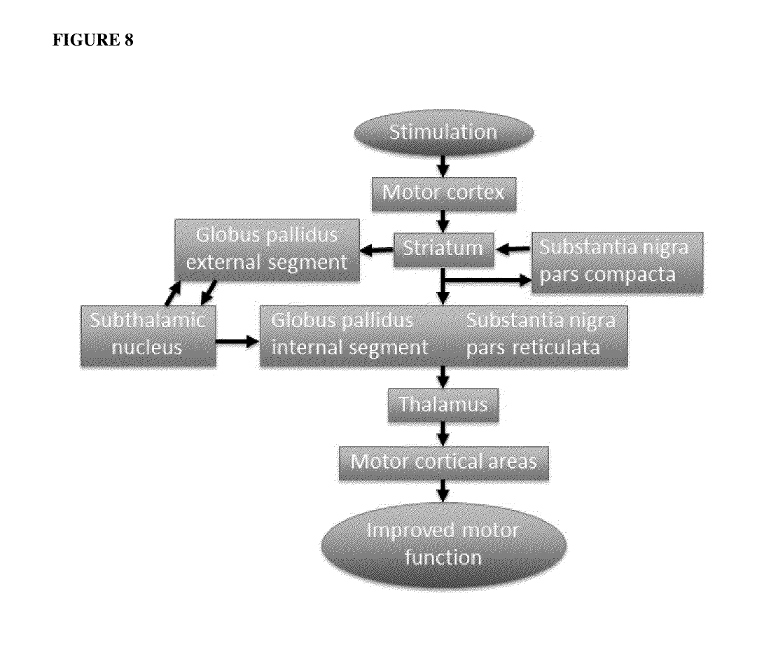

The invention generally relates to methods for improving balance of a subject. Methods of the invention focus stimulation in select regions of the central nervous system (e.g., brain or spinal cord) to modulate one or more signals sent from or received by the central nervous system. While not being limited by any particular theory or mechanism of action, properly dosed non-invasive brain stimulation (NIBS) techniques can effectively treat chronic diseases, and work by inducing therapeutic, long-lasting neuroplastic effects in disease affected brain targets. In fact, the neuromodulatory effects of NIBS can outlast the duration of transient stimulation, as has been demonstrated with electrophysiology recordings, imaging methods, pharmacologic studies, metabolic measurements, and clinical measures in both animals and humans. While the after-effects from a single transient session of NIBS are usually short lived (minutes to hours), repeated NIBS transient sessions (e.g., separate sessions on consecutive days) have been shown to alter brain activity for periods lasting months after stimulation ends (dependent on NIBS type, dose, brain target, and disease state). In many cases, the cumulative effects of NIBS can be further maintained with additional stimulation sessions, applied at the time when the neuromodulatory effects begin to wane. Effective NIBS therapeutic effects can be characterized accordingly with an interaction model, in which cumulative sessions of NIBS are used to induce neuroplasticity that facilitates brain compensatory mechanisms (or suppress abnormal activity) and improve patient function in the presence of disease. According to the interaction model in Parkinson's disease, cumulative NIBS sessions induce neuroplasticity in brain circuits affected by Parkinson's disease and revert some of the maladaptive compensatory plasticity of the disease. In the Parkinson's disease treatment example of FIG. 8, cortical NIBS to the patient's primary motor cortex, directly affects the cortical dysfunction of Parkinson's disease and can further modify activity in the dysfunctional basal ganglia networks through glutamatergic corticostriatal and cortico subthalamic projections, through this direct stimulation and transynaptic effects of stimulation balance and gait disturbances can be improved (such methods, where stimulating cortical motor areas (e.g., primary motor cortex, supplementary motor area, premotor area), could have a greater impact on axial motor signs compared to invasive DBS methodologies (implanted in basal ganglia related structures such as the STN and globus pallidus) due to different stimulation targets and/or mechanisms). In certain embodiments, by using a method like electrosonic stimulation (i.e., electrical and ultrasonic fields used for stimulation) one can stimulate deeper and more focally than conventional NIBS methods (e.g., TMS and tDCS), with greater durations and magnitudes of effects, can be more effective in improving the motor symptoms of Parkinson's disease, including gait and balance instabilities. Electrosonic stimulation can reach deeper targets and be more focal in its stimulation of targets compared to other NIBS methods and thus improve Parkinson's disease patients more effectively. NIBS Parkinson's disease treatments can be used adjunctive to physical/medical therapies, earlier in the care continuum than surgical DBS implantations, in which the lasting neuroplasticity effects of repeated NIBS sessions can be an effective intervention to ameliorate patient symptoms and improve their quality of life.

While discussed in the context of Parkinson's disease, the skilled artisan will appreciate that the methods described herein are applicable to any type of movement disorder. Exemplary movement disorders in addition to Parkinson's disease include Parkinsonism (aka., Parkinsonianism which includes Parkinson's Plus disorders such as Progressive Supranuclear Palsy, Multiple Systems Atrophy, and/or Corticobasal syndrome and/or Cortical-basal ganglionic degeneration), tauopathies, synucleinopathies, Dementia with Lewy bodies, Dystonia, Cerebral Palsy, Bradykinesia, Chorea, Huntington's Disease, Ataxia, Tremor, Essential Tremor, Myoclonus, tics, Tourette Syndrome, Restless Leg Syndrome, or Stiff Person Syndrome. Methods of the invention can also be used to improve balance of people returning from space.

Stimulation for balance improvement can be used to improve any aspect of balance such as for example gait, posture, fall likelihood, and/or coordination. Stimulation can also be used to affect the sensory receptors or their circuits so to impact balance, such as for example proprioception, visual, vestibular, or cutaneous sensory receptors or attached networks. Furthermore, stimulation for balance improvement could be used to impact the reflex networks that are part of balance function. For example, neural circuitry exists to make the flexor reflex adaptive. Because the weight of the body is supported by both legs, the flexor reflex must coordinate the activity not only of the leg being withdrawn (such as when a person steps on a sharp object) but also of the opposite leg. For example, when stepping on a sharp object with the right foot, a person will have a flexor reflex to withdraw your right leg immediately. The left leg must simultaneously extend in order to support the body weight that would have been supported by the right leg. Without this coordination of the two legs, the shift in body mass would cause a loss of balance. Thus, the flexor reflex incorporates a crossed extension reflex. A branch of the Group III afferent innervates an excitatory interneuron that sends its axon across the midline into the contralateral spinal cord. There it excites the alpha motor neurons that innervate the extensor muscles of the opposite leg, allowing balance and body posture to be maintained. Stimulation can be provided to the peripheral neurons that are connected to the circuits that affect these reflexes, directly to peripheral neurons that are part of this reflex arc, centrally to neurons connected to these circuits, and/or directly to central nervous system neurons that are part of the circuit. Another example of a reflex which impacts balance is the myotatic reflex, which for example can impact the maintenance of posture. If one is standing upright and starts to sway to the left, muscles in the legs and torso are stretched, activating the myotatic reflex to counteract the sway. In this way, the higher levels of the motor system can send commands to maintain current posture and then allow lower levels of the system to respond to the commands. The lower levels of the hierarchy implement the command with such mechanisms as the myotatic reflex, freeing the higher levels to perform other tasks such as planning the next sequence of movements. As in the flexor and crossed extensor reflexes, for the myotatic reflex stimulation can be provided to the peripheral neurons that are connected to the circuits that affect these reflexes, directly to peripheral neurons that are part of this reflex arc, centrally to neurons connected to these circuits, and/or directly to central nervous system neurons that are part of the circuit. Other examples of reflex neural circuits which are part of balance include transcortical long-loop reflexes, transcortical reflexes, postural reflexes, primitive reflexes, cervicoocular reflex, cervicospinal reflex, somatosensory reflexes, vestibulocollic reflexes, vestibulospinal reflexes, tonic neck reflexes, vestibular spinal reflexes, labyrinthine reflexes, optical righting reflexes, placing reactions, hopping reactions, and/or righting reflexes.

Furthermore, under certain conditions when patients are suffering from chronic injuries, such as a trauma-induced injury, they can develop compensatory mechanisms to function in normal activities of daily life. These compensatory mechanisms can lead to an alteration of neural network activity that can impact balance, and thus by targeting these neural networks of balance (directly and/or through trans-synaptic connections) stimulation can improve balance in these patients.

Any type of noninvasive stimulation known in the art may be used with methods of the invention, and the stimulation may be provided in any clinically acceptable manner. Exemplary types of noninvasive stimulation include mechanical, optical, electromagnetic, thermal, or a combination thereof. In particular embodiments, the stimulation is a mechanical field (i.e., acoustic field), such as that produced by an ultrasound device. In other embodiments, the stimulation is an electrical field. In other embodiments, the stimulation is a magnetic field. Other exemplary types of stimulation include Transcranial Direct Current Stimulation (TDCS), Transcranial Ultrasound (TUS)/Transcranial Doppler Ultrasound (TDUS), Transcranial Electrical Stimulation (TES), Transcranial Alternating Current Stimulation (TACS), Cranial Electrical Stimulation (CES), or Transcranial Magnetic Stimulation (TMS). In other embodiments, the stimulation source may work in part through the alteration of the nervous tissue electromagnetic properties, where stimulation occurs from an electric source capable of generating an electric field across a region of tissue and a means for altering the permittivity of tissue relative to the electric field, whereby the alteration of the tissue permittivity relative to the electric field generates a displacement current in the tissue. The means for altering the permittivity may include a chemical source, optical source, mechanical source, thermal source, or electromagnetic source.

In other embodiments, the stimulation is provided by a combination of an electric field and a mechanical field. The electric field may be pulsed, time varying, pulsed a plurality of time with each pulse being for a different length of time, or time invariant. Generally, the electric source is current that has a frequency from about DC to approximately 100,000 Hz. The mechanical field may be pulsed, time varying, or pulsed a plurality of time with each pulse being for a different length of time. In certain embodiments, the electric field is a DC electric field.

In other embodiments, the stimulation is a combination of Transcranial Ultrasound (TUS) and Transcranial Direct Current Stimulation (TDCS). Such a combination allows for focality (ability to place stimulation at fixed locations); depth (ability to selectively reach deep regions of the brain); persistence (ability to maintain stimulation effect after treatment ends); and potentiation (ability to stimulate with lower levels of energy than required by TDCS alone to achieve a clinical effect).

In certain embodiments, methods of the invention focus stimulation on particular structures in the brain that are associated with Parkinson's disease or other movement disorders, such as the substantia nigra. Other structures that may be the focus of stimulation include the basal ganglia, the nucleus accumbens, the gastric nuclei, the brainstem, the inferior colliculus, the superior colliculus, the periaqueductal gray, the primary motor cortex, the premotor cortex, the supplementary motor cortex, the occipital lobe, Brodmann areas 1-48, the primary auditory cortex, the hippocampus, the cochlea, the cranial nerves, the frontal lobe, the occipital lobe, the temporal lobe, the parietal lobe, the cortex, the sub-cortical structures, and the spinal cord. Additional targets (alone or in combination with each other or any of the above targets) for stimulation (alone or in combination with each other or any of the above targets) and/or part of the stimulatory loops that can be affected to impact balance include but are not limited to specific areas such as the cerebellum (such as to impact unconscious proprioception), somatosensory cortex (such as for example stimulation could impact the generation and/or transmission of proprioceptive feedback to cerebellum), vestibular cortex (such as for example in insular areas), visual cortex (such as for example stimulation could impact the generation and/or transmission of feedback information to the cerebellum), superior aspects parietal lobe (e.g., such as for example stimulation could impact the areas responsible for visual signal synthesis), superior parietal lobule and inferior parietal lobule (e.g., such as for example stimulation could impact the areas of body or spatial awareness), pontine reticulospinal tract, pontine reticular formation, medullary reticulospinal tract, reticulospinal tract (e.g., stimulation can affect integration of sensory input that is used to guide motor output and to impact balance), tectospinal tract, vestibulospinal tract(s) and/or vestibular nuclei (for example, the vestibulospinal tracts mediate postural adjustments, mediate head movements, help the body to maintain balance and thus stimulation can be provided to impact any of these aspects of balance). Furthermore, small movements of the body are detected by the vestibular sensory neurons, and motor commands to counteract these movements are sent through the vestibulospinal tracts to appropriate muscle groups throughout the body. The lateral vestibulospinal tract excites antigravity muscles in order to exert control over postural changes necessary to compensate for tilts and movements of the body. The medial vestibulospinal tract innervates neck muscles in order to stabilize head position as one moves around the world. It is also important for the coordination of head and eye movements. And thus stimulation can be focused to any of these tracts or connected nuclei to impact balance), the rubrospinal tract, red nucleus of the midbrain, corticospinal tracts, internal capsule, crus cerebri (cerebral peduncle), medulla, brainstem, pyramids, ventral horn of spinal cord, and/r the dorsal horn of spinal cord.

The parameters of the stimulation can be tuned so that effective stimulation is provided in a short period of time, allowing for a subject to receive stimulation while awake with little disruption to their day. That is opposed to stimulation protocols that require that stimulation be provided for long periods of time while a subject sleeps, making methods of the invention safer and easier to administer than methods that are administered to a sleeping patient. For example, long periods of stimulation, for instance while a patient sleeps, can lead to risks to patients that would require constant patient monitoring, such as an EEG to assess brain state and/or the potential for overstimulation and/or stimulator contact site abnormalities (e.g., one would need a sleep state derived EEG triggered brain stimulation system for sleeping patients, that monitors what sleep stage a person is in to provide stimulation during the same brain state during subsequent periods of sleep. One would further need to use a dry electrode methodology for stimulation (if using electrode based methods, e.g., tDCS or TES), or implement a technique to monitor and keep the electrodes hydrated during long periods while a person sleeps (e.g., a sensor to monitor electrode hydration states and an automated system to provide electrodes with saline (or other electrode contact material), or an algorithm based on use and environment to determine times to provide additional electrode contact materials, and/or need an external monitor (who is awake) to evaluate and adjust the subject and stimulation set-up during long periods of wear (particularly if a patient is asleep)); similarly, one would need comparable implementations for other types of stimulation sources (for example a method to keep an ultrasonic transmission medium between a patient and sleeping subject). One would also need to implement additional safety features to the stimulator and stimulator interface to monitor the patient during long periods of stimulation (and/or long periods of wearing an inactive stimulation unit) to monitor things such as brain state (e.g., EEG to assess for abnormal EEG patterns indicative of overstimulation, such as the potential for kindling from overstimulation), skin state (e.g., long term contact irritation or abnormal contact site reactions), stimulation source state (contact state, position, etc.)).

In certain embodiments, the stimulation is provided in a single session that lasts 3 hours or less. For example, a single session may last 2.5 hours or less, 2 hours or less, 1 hour or less, thirty minutes or less, 25 minutes or less, 20 minutes or less, 15 minutes or less, 10 minutes or less, or 5 minutes or less. An exemplary stimulation protocol may involve numerous stimulation sessions over multiple days, with no single session lasting more than three hours. For example, a stimulation protocol may be two weeks in length, in which a subject received a single 20 minute session of stimulation each day of the week, or stimulation 5 days/week over a two week period (e.g., on weekdays for 10 total stimulation sessions). The stimulation can be tuned such that nothing more than stimulation of the central nervous system is required to improve the subject's balance, i.e., the methods of the invention are performed without additionally stimulating the peripheral nervous system and/or without the use of a therapeutic agent. In other embodiments, the stimulation is provided in combination with use of a therapeutic agent. In those embodiments, the stimulation enhances the efficacy of the medication and the medication and the stimulation work in combination to improve the subject's balance. In other embodiments where stimulation is provided in conjunction with a therapeutic agent, the therapeutic agent can be used to affect one part of a disease and stimulation another part of the disease, but the different effects could be used together to improve a patient's balance.

In some embodiments, stimulation may be provided and/or tuned with a therapeutic pharmacologic agent regimen and/or a single agent. For instance with Parkinson's Disease (PD), a patient could be given stimulation tuned around the patient being in the ‘on’ period of levodopa treatment (i.e., Although levodopa is an effective pharmacologic treatment for Parkinson's disease, there can be variability in an individual's response to treatment—so-called “motor fluctuations.” The fluctuating response to levodopa can be broadly described in “on” and “off” periods. During an “on” period, a person can move with relative ease often with reduced tremor and stiffness. “Off” periods describe those times when a person has greater difficulty with movement. During these periods, balance can also be impacted accordingly). See Connolly et al. (JAMA. 2014 Apr. 23-30; 311(16):1670-83. doi: 10.1001/jama.2014.3654), the content of which is incorporated by reference herein in its entirety. Stimulation can be given at any time with the drug regimen that is correlated with maximizing efficacy. For instance with PD, it may be shown that the maximum therapeutic effect of stimulation is attained by giving patients stimulation at the onset of their “On” period, or that stimulation is giving during the middle of their “On” period. Any combination of stimulation with a pharmacologic regimen for maximum efficiency can be delivered.

Stimulation can be given while the subject is awake. Stimulation that requires the subject to be in a sleep state requires the brain to be in a different electrophysiological state (e.g., during sleep, power levels in EEG bands are different than when awake) than an awake subject, and brain stimulation methods are dependent on the brain state of the targeted neural tissue to effectively alter neural function (sleep based stimulation, designed for consistent stimulation effects, should be triggered by an EEG monitor to make sure that the stimulation therapy was being given with the brain in the same state (e.g., throughout sleep stages, the brain goes through many different states)). Furthermore, the effects of stimulation in an awake patient can be different from a sleeping patient. As we have shown (see Examples), noninvasive stimulation given to an awake patient can be effective in improving a patients' balance with both a patient's eye's open and eye closed.

Furthermore, stimulation to different areas of the central nervous system (e.g., brain and/or spinal cord) can be used to affect different aspects of balance. One can provide stimulation to affect central nervous system (e.g., brain and/or spinal cord) functions that can have both a primary and/or secondary effect on balance, including for example sensory function, proprioceptive information, motor function, vestibular function, vibration function, joint position sense, body position sense, vision function, multi-modal sensory processing, and/or cortical and subcortical balance networks. Stimulation can be used to improve the processing of this type of information when pathologies result in a lack of complete information for balance processing and/or unbalanced central processing of balance information (such as for example what might result from an unbalanced neural network where certain nodes are abnormally excited or inhibited (and/or the connections between the networks)). For example, part of the brain responsible for processing tactile sensory information might be overexcited in comparison to part of the brain processing visual information whereby both pieces information cannot be used appropriately for balance processing, and in such an example brain stimulation can be used to alter the excitability of one or both of the brain areas to bring the processing of balance information back into equilibrium so that patient balance is improved.

Stimulation of the central nervous system can also be used to improve balance abnormalities that are generated from peripheral abnormalities. For instance, if a patient has a balance disturbance related to a lack balance information coming from their feet (e.g., sensory information, proprioceptive information, pressure sense, and/or vibration sense), stimulation can be used to improve the perception of balance information in the periphery, processing balance information centrally, or a differential processing based on multiple pieces of balance information. For example, a patient might not be receiving (and/or generating) sensory information from their feet due to limited sensation in their feet, which can lead to a balance abnormality, but stimulation of balance processing centers in the brain allows for a proper assessment of other balance information (e.g., visual information and other cues) where without stimulation the balance processing would not be accurately made in the central nervous system due to a lack of complete balance information for proper central processing. In this example, the stimulation allows for a rebalancing of the balance information and/or signal processing to improve balance function.

Stimulation can also be used to effect different aspects of balance control, such as balance function with the eyes open or closed (balance can be controlled differently with the eyes open and the eyes closed as there is a difference in sensory inputs (for example, one would have to rely on other information (such as proprioceptive sense) without the combination of visual input that is part of balance)). One can also provide stimulation to affect asymmetries in balance, such as for example stimulation can be provided to a particular part of the brain more in need of stimulation than others where an asymmetry in neural excitability in that brain region results in asymmetrical balance in a patient. Or one could provide a differential level of stimulation to different parts of the brain which have an asymmetry in function that results in a balance disorder, such as for example with differences between the left and right side of the body. Balance can be further affected by asymmetries in the lower limb of patients that can for example result from abnormalities in the central nervous system and/or particular parts of the system that controls the areas and/or connects to the lower limbs (such as for example that can lead to differential strengths, speeds, coordination, and/or proprioceptive information in the lower limbs).

In certain embodiments a medical imaging modality and/or stimulator localization modality may be combined with stimulation. Exemplary imaging modalities include magnetic resonance imaging (MRI), functional MRI (fMRI), ultrasound, positron emission tomography (PET), single photon emission computed tomography (SPECT), computer aided tomography scan (CAT-scan), XRAY, optical coherence tomography (OCT), diffusion tensor imaging (DTI), diffusion spectrum imaging (DSI), electro-acoustic imaging, electromagnetic based imaging, electro-encephalogram (EEG), electromyogram (EMG), EOG (electrooculography), high density EEG, spectroscopy based methods (such as near-infrared spectroscopy (NIRS)), electrocardiogram (EKG) electrical based imaging, magnetic based imaging, nuclear based imaging, optical (photonic) based imaging, mechanical based imaging, thermal based imaging, combined imaging modalities, imaging with contrast agents, imaging without contrast agents, etc. In other embodiments, physiological measurements, stimulation subject assessment measures, and/or biofeedback measures are combined with stimulation.

A stimulator localization modality could be any device and/or process that can be used to localize the stimulator placement on the subject that is to receive stimulation. A stimulator localization modality can be any device or method which localizes an anatomical target(s) (individually, in groups, and or relative to each other) relative to the stimulation placement and/or relative brain targets for stimulation. For instance the stimulator localization modality can be a measurement tool that localizes surface anatomical locations on a subject relative to a subject's nervous system and/or to desired stimulation placement (e.g., a measurement device or method to localize a stimulation site(s) on the scalp surface from anatomical landmarks and/or the 10/20 EEG-system). The stimulator localization modality may be integrated directly with the stimulation device(s) or exist as a separate device which functions with the stimulation device(s). Stimulator localization can be made simply from patient characteristics (such as determined from complex imaging modalities to stimulation subject assessments (such as simply taking measurements of a subjects' head and/or relative distances between anatomical landmarks and/or determining the relative location of a stimulation device(s)) as detailed herein. The stimulation location modality can be used on part or a whole stimulator or stimulators (e.g., if there were multiple electrodes or transducers as part of stimulation modality the stimulation location modality could be used on one, all, or part of the group of stimulator elements). Examples stimulator localization modalities are also shown in Wagner et al. (U.S. Pat. No. 8,718,758) and Wagner et al., (U.S. patent application publication number 2011/0275927), the content of each of which is incorporated by reference herein in its entirety.

The imaging modalities, stimulation localization modalities, physiological measurements, stimulation subject assessment measures, and/or biofeedback measures could be used to assist in the stimulation by aiding in the targeting (localizing) of stimulation, dosing of stimulation, characterizing safety parameters, analyzing the online or offline effects of stimulation, and/or maximizing the therapeutic effect of stimulation. This facilitation could also be done by altering or controlling the stimulation source(s), field parameters, and/or the stimulation interface apparatus parameters.

In terms of targeting tissues to stimulate, the targeted region can be imaged with any imaging modality that provides anatomical information about the region. That image could then be used to determine the placement of the stimulation source. For example, with an electrosonic (electrical source and mechanical (i.e., sonic/ultrasound), note electrosonic is used synonymously with electromechanical herein) approach one would determine the placement of the electrical source and the ultrasound source to target the desired regions, either directly or within an interface apparatus.

The imaging information could also be used to provide guidance for the design and proper tuning of an interface apparatus between the subject to be stimulated and the stimulation source(s). For example, one might simply determine the placement of the source(s) of stimulation and/or the properties of the interface apparatus between the stimulation patient and the device (such as for example the dimensions, materials impedances, and/or design criteria) based on anatomical landmarks determined from the image and predetermined source characteristics (such as for example the beam profile of an ultrasonic transducer and the predicted field distribution of an electric field source).

Additionally, the implementation of an imaging system for targeting could also be used to direct the source fields necessary for stimulation based on calculations developed from the imaging information (or to calculate the field to correlate to stimulation effects following stimulation) and/or physiological measurements, stimulation subject assessment measures, and/or biofeedback measures. An imaging modality could be used to identify the tissue distribution of the subject to be stimulated, from which tissue boundaries in the stimulation area can be identified. This tissue and/or boundary identification could be pursued with any image analysis algorithm, and could be completed prior to stimulation, during stimulation, or following stimulation.

Once the tissues are identified, a ‘computational mesh’ can be built to capture the tissue segmentation demonstrated in the images, where mesh components can be assigned any physical and/or chemical characteristic of which will be used in determining targeting and localization of the fields, chemicals, and/or stimulation effects (e.g., material properties, electromagnetic properties, thermodynamic properties, mechanical/acoustic properties, optical properties, chemical properties, etc). These properties could be assigned known values determined before stimulation, with values determined during stimulation, or with values determined following stimulation.

Following the generation of a computational mesh based on the tissue properties (and geometry) to be modeled, models can be generated with computational/numerical solvers that capture the physics and/or chemistry of the underlying system such as by also including the source and/or interface properties (position, size, shape, and/or material properties) and/or source field characteristics (amplitude, waveform (shape/timing dynamics), frequency (power components and/or pulse frequencies if using pulsed field), and/or timing information) and/or chemical agent characteristics (concentrations, distributions, compositions, kinetics, and/or additional information).

This can be used to determine the driving field's focus, orientation, focality, and overall distribution in the tissues to be stimulated (such as for example one could determine the electrical field, voltage, current density, magnetic field, force field, mechanical field (acoustic field), pressure field, tissue acceleration, tissue position, tissue velocity, tissue temperature, etc.) or the chemical reactions and/or chemistry effects that are modeled (kinetics, chemical distributions, reactions, etc) in the tissue(s) to be stimulated. For a method where tissue properties are modified relative to an applied electric field to generate a new current, this information could then be used to calculate the altered tissue electromagnetic properties (and/or relative positions) relative to the applied electrical field in the tissue(s) to be stimulated, such that one can calculate the newly generated current density and/or electrical field distributions (such calculations can be made with any particular means for altering the tissue electromagnetic properties (including but not limited to mechanical, thermal, electromagnetic, and optical means) in the tissue(s) to be stimulated. Additionally, this information could also be used to guide the placement, design, material properties, and/or modification of an interface mechanism.

Ultimately this can allow for pre, during, or post stimulation targeting/localization via calculations based on the initial imaging modality, tissue characteristics, field source characteristics, and/or the properties of the interface apparatus (and/or the source characteristics of the means that alters the electromagnetic properties of the tissue to be stimulated from combined methods where new currents are generated relative to an electric field source). These methods could be implemented with any form of stimulation, including but not limited to electromagnetic, mechanical (i.e., acoustic), optical, thermal, electrical, magnetic, and/or combined methods (and/or methods which alter tissue impedances relative to electrical sources to generate altered stimulation currents, for example with electromagnetic, mechanical (i.e., acoustic), optical, thermal, electrical, magnetic, and/or combined sources).

In one particular example, in the area of brain stimulation, with an electrical source generating an applied electrical field and/or ultrasound (i.e, mechanical) source generating focused acoustic energy on the tissue area to be stimulated, the electrical field distribution and/or the mechanical field distribution can be calculated based on the relative electrical field and mechanical field transducer source characteristics (transducer position(s), transducer size(s), transducer shape(s), field frequencies, field time dynamics, field amplitudes, field phase information, etc) to anatomical tissue distribution (with the appropriate tissue characteristics (for example the electromagnetic properties and tissue mechanical/acoustic properties)) which can be determined from any imaging methodology which provides anatomical information about the area to be stimulated (such as for example a CAT-scan and/or MRI) and/or with predetermined tissue characteristics (and/or also with values which at least in part could be determined via an imaging modality, such as conductivity characteristics based on DTI images); for example one might solve a modified Laplacian,

$$∇ \cdot ( \frac{\partial ( ɛ ∇ \overline{Φ} )}{\partial t} + σ ∇ \overline{Φ} ) = 0 ,$$

for the an electrical potential (where Φ is solved in the sinusoidal steady state for particular angular frequency, ω, of the electrical source for particular permittivities, ∈, and conductivities, σ, of the tissues being examined (as functions of the frequency of the stimulation electrical field)) based on a particular electrical source, and the Westervelt equation:

$${∇}^{2} p - \frac{1}{{c}^{2}} \frac{{\partial }^{2} p}{\partial {t}^{2}} + \frac{δ}{{c}^{4}} \frac{{\partial }^{3} p}{\partial {t}^{3}} + \frac{β}{ρ {c}^{4}} [ p \frac{{\partial }^{2} p}{\partial {t}^{2}} + {( \frac{\partial p}{\partial t} )}^{2} ] - ∇ p \cdot ∇ ( ln p ) = 0$$

for a particular mechanical source (where p is pressure, and c is the speed of sound, δ is acoustic diffusivity, β is the coefficient of nonlinearity, and ρ is the density of the respective tissues), and the appropriate boundary conditions between varied tissues. The calculated electrical and mechanical field distributions can be used to calculate the altered tissue electromagnetic properties (and/or relative tissue positions (with varied tissue electromagnetic properties)) relative to the applied electrical field, such that one can calculate the newly generated current density and/or electrical field distributions; for example one could pursue tissue/field perturbation model and/or a hybrid Hinch/Fixman (Fixman et al., J Chem Phys. 1980; 72(9):5177-86; Fixman et al., J Chem Phys. 1982; 78(3):1483-92; Hinch et al., J Chem Soc, Farady Tans. 1983; 80:535-51; Chew J Chem Phys. 1984; 80(9):4541-52; and Chew, J Chem Phys. 1982; 77:4683) inspired model of dielectric enhancement to determine field perturbations and changes in bulk permittivity, thus ultimately calculating the current density distributions in the brain during stimulation (where J=σE+∂(∈E)/∂t, J is the current in the tissue, σ the tissue conductivity, E the total field (i.e., source plus perturbation field), and ∈ is the tissue permittivity; in regions outside of the main focus fields could be determined through continuity equations).

This information will in turn allow one to predict the distribution of the fields and/or currents in the brain based on the imaging and stimulation source information and thus predict locations of effect of stimulation (and/or magnitude of effect). If one chose to use an interface apparatus during the stimulation, such as a helmet like mechanism, the helmet itself could be tailored uniquely for a subject being stimulated based on the calculated field and/or targeting information (such as where one could integrate the helmet design and materials into all of the subsequent physics (and chemical) based calculations). This information and/or resulting calculations could also be integrated with physiological measurements, stimulation subject assessment measures, and/or biofeedback measures, as it could be used to assist in the stimulation by aiding in the targeting (localizing) of stimulation, dosing of stimulation, characterizing safety parameters, and/or analyzing the online or offline effects of stimulation. This facilitation can also be done by altering or controlling the stimulation source(s), field parameters, and/or the stimulation interface apparatus parameters (based on the calculations and/or other feedback information).

One could implement a closed loop system which could automatically tune stimulation based on the calculations and/or feedback which is gathered and fed into an automated control system(s) to tune stimulation results to a desired response based on a particular algorithm and/or an adaptive system; one could implement a system which allows a person or persons operating the stimulation system to modify the stimulation system itself to achieve a desired response relative to the information/feedback that is gathered; and/or a hybrid system of control (note that the information/feedback can be gained from any imaging modalities, biofeedback, physiological measures, and/or other measures as exemplified above). Accordingly, these methods could be implemented with any stimulation method by adapting the physical field calculations appropriately (for example electrical field sources and effects could be calculated with the modified Laplacian equation or TUS acoustic fields could be solved with the Westervelt equation alone (one could also calculate local field changes based on sources of electrical fields such charged proteins, membranes, and macromolecules, similar to the methods outlined above).

These methods could be implemented with any form of stimulation. Exemplary types of stimulation include mechanical, optical, electromagnetic, thermal, or a combination thereof. In particular embodiments, the stimulation is a mechanical field (i.e., acoustic field), such as that produced by an ultrasound device. In other embodiments, the stimulation is an electrical field. In other embodiments, the stimulation is a magnetic field. Other exemplary types of stimulation include Transcranial Direct Current Stimulation (TDCS), Transcranial Ultrasound (TUS)/Transcranial Doppler Ultrasound (TDUS), Transcranial Electrical Stimulation (TES), Transcranial Alternating Current Stimulation (TACS), Cranial Electrical Stimulation (CES), or Transcranial Magnetic Stimulation (TMS). In other embodiments, the stimulation source may work in part through the alteration of the nervous tissue electromagnetic properties, where stimulation occurs from an electric source capable of generating an electric field across a region of tissue and a means for altering the permittivity of tissue relative to the electric field, whereby the alteration of the tissue permittivity relative to the electric field generates a displacement current in the tissue. The means for altering the permittivity may include a chemical source, optical source, mechanical source, thermal source, or electromagnetic source.

Stimulation targeting, localization, and/or field information could also be integrated with additional technologies. For instance, one could integrate the imaging based field solver methodologies with frameless stereotactic systems to track/target stimulation location during a procedure. Additionally, as this targeting, localization, and/or field information can be used to predict the strength and orientation of the current densities (and/or other fields) generated in the tissues relative to the tissue to be stimulated, this information can in turn be fed into neural modeling algorithms (such as Hodgkin and Huxley based stimulation models) that can be used to predict the neural response and/or the information can be used to guide dosing of stimulation. Additionally, the information could be used to adjust the parameters of stimulation and or the characteristics of the interface.

Imaging modalities, physiological measurements, stimulation subject assessment measures, and/or biofeedback measures can also be used to track the effect of stimulation, and ultimately be integrated with the stimulator and/or a interface apparatus to provide a closed loop system of controlled stimulation (and/or with the targeting/field information described above). Imaging modalities that provide information such as but not limited to tissue electrical activity (such as for example, EEG data from the brain for neural stimulation or EKG information from the heart for cardiac stimulation or EMG data from muscle during neural and/or muscle stimulation or electro-retinal gram (ERG) data for visual system stimulation), tissue metabolic information (such as from glucose information from a fluorodeoxyglucose (FDG) based PET scan), tissue blood flow/absorption (such as blood flow information that might be determined from a BOLD signal that might be determined during MRI or with modified functional measures), neuroreceptor activation (such as through radioligands that bind to dopamine receptors and can be imaged with modalities such as PET), tissue temperature changes (such as from thermal imaging), and/or any information of tissue response could be integrated with the stimulation method to provide system based feedback and provide guidance to hone stimulation field parameters such as the stimulation duration, stimulation waveform shape (amplitude and dynamics); source position, size, shape relative to tissues to be stimulated; and/or stimulation targeting, localization, and/or field parameters, such as the source fields timing dynamics, amplitude and orientation. Such imaging modalities, used to track the effect of stimulation, could also be integrated with methods elaborated on above to assist in targeting and dosing calculations.

Similarly physiological measurements such as but not limited to heart rate, respiratory rate, blood gas levels, blood pressure, respiratory gas compositions, urine and fluid concentrations, blood chemistry (including hormone levels), electrolyte levels, pain markers, stress indicators, joint function measures (e.g., mobility, strength, range of motion), patient weight, sensory markers, auditory measures, perceptual measures, emotional markers, skin conductance (i.e., sweat level), pupil dilation, emotional markers, temperature, fluid levels, body/limb position, fatigue markers, fear markers, coordination measures, psychiatric markers, addiction markers, motor performance measures, and/or eye position/movement could be also integrated with the stimulation method to provide system based feedback and provide guidance to hone stimulation field parameters such as the stimulation duration, stimulation waveform shape (amplitude and dynamics); source position, size, shape relative to tissues to be stimulated (e.g., shape and position of stimulation sources relative to the shape and position of the tissues to be stimulated); and/or stimulation targeting, localization, and/or field parameters, such as the source fields timing dynamics, amplitude and orientation.

Such physiological measurements, used to track the effect of stimulation, could also be integrated with methods elaborated on above to assist in targeting and dosing calculations. Additionally, one could use other biofeedback or stimulation subject assessment information directly gathered from the subject being stimulated such as but not limited to task performance (such as a motor performance, memory, or learning task), subject response (such as to depression based questionnaire/metrics to assess mood), pain measures (such as pain assessment levels or amount of pain killers used), addiction measures (such as alcohol consumption or drug use), subject gathered reports, subject based observations, and/or any subject based self-assessments could be also integrated with the stimulation method to provide system based feedback and provide guidance to hone stimulation field parameters such as the stimulation duration, stimulation waveform shape (amplitude and dynamics); source position, size, shape relative to tissues to be stimulated; and/or stimulation targeting, localization, and/or field parameters, such as the source fields timing dynamics, amplitude and orientation. Such measures, used to track the effect of stimulation, can also be integrated with methods elaborated on above to assist in targeting and dosing calculations.

One could tune/adjust such things as the stimulation source(s) position(s), size(s), and/or shape(s) relative to the tissue to be stimulated (such as the electrodes for generating the electric fields, transducers for generating acoustic fields, and/or the source of the means for modifying the electromagnetic parameters of tissues to be stimulated (i.e., mechanical/acoustic field source/transducer, optical source, thermal source, chemical source, and/or a secondary electromagnetic field source)); the field(s) that are generated from sources in terms of magnitude, direction, waveform dynamics, frequency characteristics (power spectrum of waveform and/or potential pulse frequency of stimulation field waveforms), phase information, and/or the duration of application; and/or chemical processes (duration, kinetics, chemical concentrations, distributions, etc) driven by sources.

Additionally, imaging modalities, physiological measures, biofeedback measures, stimulation subject assessments, and/or other measures might not just be integrated with the process that stimulates tissues through the combined application of electrical and/or mechanical fields (and/or chemical agents, thermal fields, optical fields/beams, and/or secondary electromagnetic fields), but effectively they could also be integrated with an interfacing apparatus to increase the interface apparatus's efficiency or modify its use relative to the measures outlined above such as but not limited to altering the material properties of the interface (such as for example altering the electrical impedance of a component(s) of the interface or altering a mechanical/acoustic properties of a component(s) of the interface mechanism such as the acoustic impendence); alter the interface apparatus position, size, shape, and/or position; alter the components of the stimulation process that it stores or interfaces with (such as in size, shape, and/or position; for example the source of the electric field and/or means to alter the tissue electromagnetic properties for tissue stimulation); altering composition(s) of the material(s) within and/or on the interface (such as fluid concentrations to couple a mechanical source with tissues to be stimulated); to control the number of uses of the interface (or the duration of its use); and/or any adjustable quality as described above in the interface description.

These modifications can be made before a stimulation session (based on previously obtained/analyzed information), during stimulation (with real time or online information), or following stimulation for subsequent stimulation sessions (with data analyzed following stimulation). One could also adjust/tune the stimulation parameters based on the information acquired before stimulation not compared to anything, during stimulation (online) compared to the pre-stimulation baseline, inter-stimulation session comparisons, cross stimulation session comparisons, pre vs. post stimulation comparisons, across multiple samples (such as across patient populations with averaged data), and/or any combination or permutation in which the data is obtained and/or analyzed. These methods could be implemented with any form of stimulation, including but not limited to electromagnetic, acoustic, optical, thermal, electrical, magnetic, and/or combined methods (and/or methods which alter tissue impedances relative to electrical sources to generate altered stimulation currents, for example with electromagnetic, acoustic, optical, thermal, electrical, magnetic, and/or combined sources).

One could implement a closed loop system which could automatically tune stimulation based on the information/feedback which is gathered and fed into an automated control system(s) to tune stimulation results to a desired response based on a particular algorithm and/or an adaptive system; one could implement a system which allows a person or persons operating the stimulation system to modify the stimulation system itself to achieve a desired response relative to the information/feedback that is gathered; and/or a hybrid system of control (note that the information/feedback can be gained from any imaging modalities, biofeedback, physiological measures, and/or other measures as exemplified above).

For example in the area of brain stimulation, with an electromechanical (i.e., electrosonic) based stimulator, with an electrical source providing a primary electric field and an acoustic source providing focused acoustic energy, one could set up a system such that source electrodes for generating the primary electric field can have their size, shape, and/or position modified in real time as directed by imaging information (and/or any other type of information) that is being gathered during stimulation. Similarly, one could set up a system such that a source transducer for generating an acoustic field can have its shape (and/or size) modified in real time and/or have its position changed in real time as guided by imaging information (and/or any other type of information) that is being gathered during stimulation.

Similarly the fields that are generated by these sources can have their amplitude, waveform dynamics/timing, frequency characteristics, phase characteristics, distribution, duration, direction, and/or orientation altered as directed by imaging information (and/or any other type of information) that is being gathered before, during, or after stimulation. Similarly if an interface apparatus is being used, it could have any of characteristics altered (size, shape, position, material properties, source contained positions (sizes and/or shapes), etc), such as for example part of its electrical impedance altered such that an electrical field that is targeting underlying tissue could be redirected to another tissue location as guided by imaging information (and/or any other type of information) that is being gathered during stimulation. For example, one could provide electromechanical stimulation (electrical field combined with a mechanical field) to a subject's brain while simultaneously recording the EEG response, and subsequently use the EEG imaging information as a guide to neural response to guide an algorithm which controls the alteration the electromechanical stimulation parameters (for example the source position, field amplitudes, stimulation waveform, stimulation duration, etc) of the electrical and mechanical field sources to tune the desired EEG response (For example one could analyze the power and/or frequency information in the EEG signal relative to stimulation provided, and in turn adjust the stimulation parameters relative to the EEG signal (such as for example, the amplitude and/or frequency properties of the mechanical and electrical source generated fields could be adjusted relative to the real time EEG response).

Or for example, one could adjust the location of the source positions along a stimulation subject's scalp, based on field calculations made as explained above, but additionally tuned with functional MRI (fMRI) information depicting location effects of stimulation, and further integrated with real time EEG data). The stimulation parameters could simply be modified by a person administering the stimulation, or be automatically controlled through a computer/machine based feedback control system during stimulation (essentially making a closed loop system), and/or a hybrid system of control. Or furthermore, the interface between the electrical field source and/or the acoustic field source could be modified through the controlled feedback system to aid in targeting or to optimize the therapeutic effect of stimulation.

Additionally, imaging modalities, physiological measures, biofeedback measures, stimulation subject assessments, and/or other measures might also be used to monitor safety parameters in the tissue before, during, and/or after stimulation (either via calculations based on the imaging and source information, and/or measured information alone). For instance one could use the thermal information to assure tissue temperatures remain within desired levels, electrical activity information to assess for potential seizure activity or abnormal neural response, current density magnitude calculations in the tissue (including a breakdown of the current types (i.e., ohmic vs. capacitive)) to determine if stimulation currents are within appropriate safety windows, psychological measures from a stimulation subject response (such as for example markers for depression and/or mood) to determine if stimulation is having the appropriate psychological response, physiological measures from a stimulation subject (such as for example heart rate and other system measures) to determine if stimulation parameters are being applied safely, and/or other various safety markers.

These different methods can all be combined together in whole or in part and used to tune and/or alter the stimulation source characteristics, field parameters, calculated fields, the interface apparatus characteristics, and/or other qualities at any point before, during, or after stimulation to aid in the targeting (localizing) of stimulation, dosing of stimulation, characterizing safety parameters, and/or analyzing the online or offline effects of stimulation.

And furthermore, such imaging, biofeedback, physiological measurement, and other modalities in conjunction with the altered current generation could similarly be applied in the areas of altering cellular metabolism, physical therapy, drug delivery, and gene therapy as explained in the referenced patent application (U.S. patent application Ser. No. 11/764,468, Apparatus and Method for Stimulation of Biological Tissue) and above as focused on treating Osteoarthritis (OA). These examples are provided not to be exhaustive, but as an example of potential applications. Furthermore, stimulation for improving balance, posture, and/or gait can be coupled with physical therapy based training, such as for example balance training. Stimulation can be provided, during, and/or after physical training. Stimulation can be timed to maximize the effects of physical training (or vice versa), such as for example the effects of certain stimulation paradigms might lead to a maximum effect at a period of time after stimulation is completed, and thus physical training could be designed to be most challenging during the period when stimulation effects are maximized, thus increasing the effects of both therapies.

All of the methods and processes discussed in this document could be implemented with any form of stimulation, including but not limited to electromagnetic, acoustic, optical, thermal, electrical, magnetic, and/or combined methods (and/or methods which alter tissue impedances relative to electrical sources to generate altered stimulation currents, for example with electromagnetic, chemical, acoustic, optical, thermal, electrical, magnetic, and/or combined sources).

In particular embodiments, the stimulation is a combination of an electric field and a mechanical field. Such a form of stimulation is described for example in Wagner et al. (U.S. patent application publication number 2008/0046053), the content of which is incorporated by reference herein in its entirety.

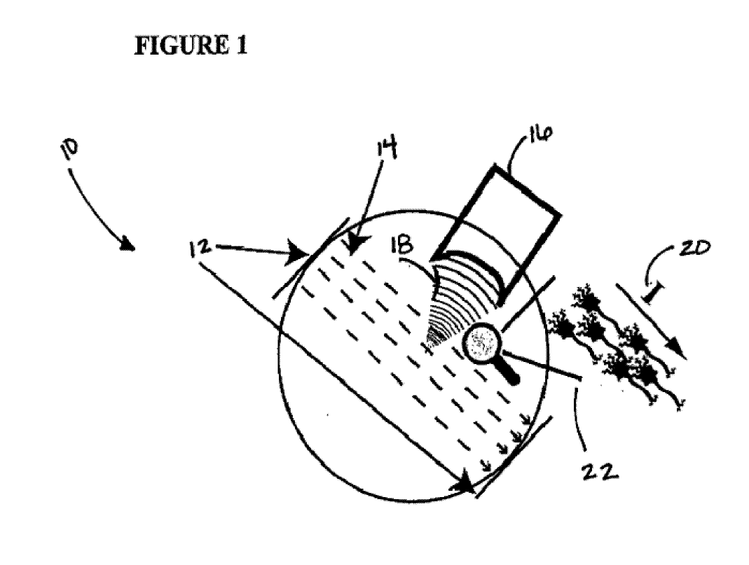

Turning now to FIG. 1, which illustrates an exemplary embodiment of an apparatus 10 to alter currents, e.g., amplify, focus, alter direction, and/or attenuate in the presence of an applied electric field or applied current source by the combined application of a mechanical field within a biological material to stimulate the biological cells and/or tissue in accordance with the present disclosure. For example, the apparatus 10 illustrated in FIG. 1 according to the present disclosure may be applied to the area of neural stimulation. An initial source electric field 14 results in a current in the tissue. The electric field 14 is created by an electric source, current or voltage source. As described in further detail below, the permittivity of the tissue is altered relative to the electric field, for example by a mechanical field, thereby generating an additional displacement current.

Electrodes 12 are applied to the scalp and generate a low magnitude electric field 14 over a large brain region. While electrodes 12 are used and applied to the scalp in this exemplary embodiment, it is envisioned that the electrodes may be applied to a number of different areas on the body including areas around the scalp. It is also envisioned that one electrode may be placed proximal to the tissue being stimulated and the other distant, such as one electrode on the scalp and one on the thorax. It is further envisioned that electric source could be mono-polar with just a single electrode, or multi-polar with multiple electrodes. Similarly, the electric source may be applied to tissue via any medically acceptable medium. It is also envisioned that means could be used where the electric source does not need to be in direct contact with the tissue, such as for example, inductive magnetic sources where the entire tissue region is placed within a large solenoid generating magnetic fields or near a coil generating magnetic fields, where the magnetic fields induce electric currents in the tissue.

The electric source may be direct current (DC) or alternating current (AC) and may be applied inside or outside the tissue of interest. Additionally, the source may be time varying. Similarly, the source may be pulsed and may be comprised of time varying pulse forms. The source may be an impulse. Also, the source according to the present disclosure may be intermittent.

A mechanical source such as an ultrasound source 16 is applied on the scalp and provides concentrated acoustic energy 18, i.e., mechanical field to a focused region of neural tissue, affecting a smaller number of neurons 22 than affected by the electric field 14, by the mechanical field 18 altering the tissue permittivity relative to the applied electric field 14, and thereby generating the altered current 20. The mechanical source may be any acoustic source such as an ultrasound device. Generally, such device may be a device composed of electromechanical transducers capable of converting an electrical signal to mechanical energy such as those containing piezoelectric materials, a device composed of electromechanical transducers capable of converting an electrical signal to mechanical energy such as those in an acoustic speaker that implement electromagnets, a device in which the mechanical source is coupled to a separate mechanical apparatus that drives the system, or any similar device capable of converting chemical, plasma, electrical, nuclear, or thermal energy to mechanical energy and generating a mechanical field.

Furthermore, the mechanical field could be generated via an ultrasound transducer that could be used for imaging tissue. The mechanical field may be coupled to tissue via a bridging medium, such as a container of saline to assist in the focusing or through gels and/or pastes which alter the acoustic impedance between the mechanical source and the tissue. The mechanical field may be time varying, pulsed, an impulse, or may be comprised of time varying pulse forms. It is envisioned that the mechanical source may be applied inside or outside of the tissue of interest. There are no limitations as to the frequencies that can be applied via the mechanical source, however, exemplary mechanical field frequencies range from the sub kHZ to 1000s of MHz. Additionally, multiple transducers providing multiple mechanical fields with similar or differing frequencies, and/or similar or different mechanical field waveforms may be used—such as in an array of sources like those used in focused ultrasound arrays. Similarly, multiple varied electric fields could also be applied. The combined fields, electric and mechanical, may be controlled intermittently to cause specific patterns of spiking activity or alterations in neural excitability. For example, the device may produce a periodic signal at a fixed frequency, or high frequency signals at a pulsed frequency to cause stimulation at pulse frequencies shown to be effective in treating numerous pathologies. Such stimulation waveforms may be those implemented in rapid or theta burst TMS treatments, deep brain stimulation treatments, epidural brain stimulation treatments, spinal cord stimulation treatments, or for peripheral electrical stimulation nerve treatments. The ultrasound source may be placed at any location relative to the electrode locations, i.e., within, on top of, below, or outside the same location as the electrodes as long as components of the electric field and mechanical field are in the same region. The locations of the sources should be relative to each other such that the fields intersect relative to the tissue and cells to be stimulated, or to direct the current alteration relative to the cellular components being stimulated.

The apparatus and method according to the present disclosure generates capacitive currents via permittivity alterations, which can be significant in magnitude, especially in the presence of low frequency applied electric fields. Tissue permittivities in biological tissues are much higher than most other non-biological materials, especially for low frequency applied electric fields where the penetration depths of electric fields are highest. This is because the permittivity is inversely related to the frequency of the applied electric field, such that the tissue permittivity magnitude is higher with lower frequencies. For example, for electric field frequencies below 100,000 Hz, brain tissue has permittivity magnitudes as high as or greater than 10̂8 (100,000,000) times the permittivity of free space (8.854*10̂−12 farad per meter), and as such, minimal local perturbations of the relative magnitude can lead to significant displacement current generation. As the frequency of the electric field increases, the relative permittivity decreases by orders of magnitude, dropping to magnitudes of approximately 10̂3 times the permittivity of free space (8.854*10̂-12 farad per meter) for electric field frequencies of approximately 100,000 Hz. Additionally, by not being constrained to higher electric field frequencies, the method according to the present disclosure is an advantageous method for stimulating biological tissue due to lowered penetration depth limitations and thus lowered field strength requirements. Additionally, because displacement currents are generated in the area of the permittivity change, focusing can be accomplished via the ultrasound alone. For example, to generate capacitive currents via a permittivity perturbation relative to an applied electric field as described above, broad DC or a low frequency electric source field well below the cellular stimulation threshold is applied to a brain region but stimulation effects are locally focused in a smaller region by altering the tissue permittivity in the focused region of a mechanical field generated by a mechanical source such as an ultrasound source. This could be done noninvasively with the electrodes and the ultrasound device both placed on the scalp surface such that the fields penetrate the tissue surrounding the brain region and intersect in the targeted brain location, or with one or both of the electrodes and/or the ultrasound device implanted below the scalp surface (in the brain or any of the surrounding tissue) such that the fields intersect in the targeted region.

A displacement current is generated by the modification of the permittivity in the presence of the sub threshold electric field and provides a stimulatory signal. In addition to the main permittivity change that occurs in the tissues, which is responsible for stimulation (i.e., the generation of the altered currents for stimulation), a conductivity change could also occur in the tissue, which secondarily alters the ohmic component of the currents. In a further embodiment, the displacement current generation and altered ohmic current components may combine for stimulation. Generally, tissue conductivities vary slightly as a function of the applied electric field frequency over the DC to 100,000 Hz frequency range, but not to the same degree as the permittivities, and increase with the increasing frequency of the applied electric field. Additionally in biological tissues, unlike other materials, the conductivity and permittivity do not show a simple one-to-one relationship as a function of the applied electric field frequency. The permittivity ranges are as discussed above.

Although the process described may be accomplished at any frequency of the applied electric field, the method in an exemplary embodiment is applied with lower frequency applied electric fields due to the fact the permittivity magnitudes of tissues, as high as or greater than 10̂8 times the permittivity of free space, and the electric field penetration depths are highest for low frequency applied electric fields. Higher frequency applied electric fields may be less desirable as they will require greater radiation power to penetrate the tissue and/or a more pronounced mechanical source for permittivity alteration to achieve the same relative tissue permittivity change, i.e., at higher applied electric field frequencies the permittivity of the tissue is lower and as such would need a greater overall perturbation to have the same overall change in permittivity of a tissue as at a lower frequency. Applied electric field frequencies in the range of DC to approximately 100,000 Hz frequencies are advantageous due to the high tissue permittivity in this frequency band and the high penetration depth for biological tissues at these frequencies. In this band, tissues are within the so called ‘alpha dispersion band’ where relative tissue permittivity magnitudes are maximally elevated (i.e., as high as or greater than 10̂8 times the permittivity of free space). Frequencies above approximately 100,000 to 1,000,000 Hz for the applied electric fields are still applicable for the method described in generating displacement currents for the stimulation of biologic cells and tissue, however, both the tissue permittivity and penetration depth are limited for biological tissues in this band compared to the previous band but displacement currents of sufficient magnitude can still be generated for some applications. In this range, the magnitude of the applied electric field will likely need to be increased, or the method used to alter the permittivity relative to the applied electric field increased to bring about a greater permittivity change, relative to the tissue's permittivity magnitude for the applied electric field frequency. Additionally, due to potential safety concerns for some applications, it may be necessary to limit the time of application of the fields or to pulse the fields, as opposed to the continuous application that is possible in the prior band. For tissues or applications where the safety concerns preclude the technique in deeper tissues, the technique could still be applied in more superficial applications in a noninvasive manner or via an invasive method. Higher frequency applied electric fields, above 1,000,000 to 100,000,000 Hz, could be used in generating displacement currents for the stimulation of biologic cells and tissue. However, this would require a more sufficient permittivity alteration or electromagnetic radiation, and as such is less than ideal in terms of safety than the earlier bands. For frequencies of the applied electric field above 100,000,000 Hz, biologic cell and tissue stimulation may still be possible, but may be limited for specialized applications that require less significant displacement currents.

In the focused region of tissue to which the mechanical fields are delivered, the excitability of individual neurons can be heightened to the point that the neurons can be stimulated by the combined fields, or be affected such as to cause or amplify the alteration of the neural excitability caused by the altered currents, either through an increase or decrease in the excitability of the neurons. This alteration of neural excitability can last past the duration of stimulation and thus be used as a basis to provide lasting treatment. Additionally, the combined fields can be provided in multiple, but separate sessions to have a summed, or carry-over effect, on the excitability of the cells and tissue. The combined fields can be provided prior to another form of stimulation, to prime the tissue making it more or less susceptible to alternate, follow-up forms of stimulation. Furthermore, the combined fields can be provided after an alternate form of stimulation, where the alternate form of stimulation is used to prime the tissue to make it more or less susceptible to the form of stimulation disclosed herein. Furthermore, the combined fields could be applied for a chronic period of time.

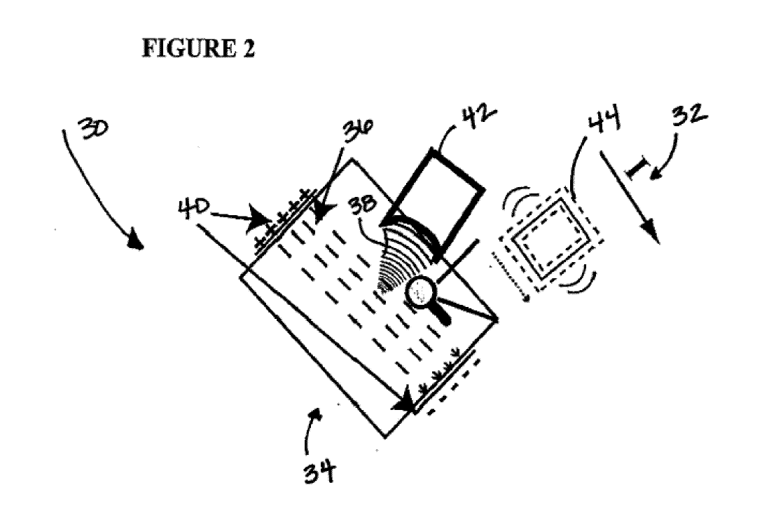

FIG. 2 illustrates a set up 30 to perform a method for generating an altered current with a newly generated displacement current 32 for stimulation in biologic tissue 34 through the combined effects of an electric field 36 and a mechanical field 38. A tissue or composite of tissues 34 is placed adjacent to the anode and cathode of an electric source 40 which generates an electric field 36. The electric field 36 is combined with a mechanical, e.g., ultrasound field 38 which can be focused on the tissue 34 and generated via an ultrasound transducer 42. In a sub-region of tissue 44 where the mechanical field 38 is focused and intersects with the electric field 36, a displacement current 32 is generated. By vibrating and/or mechanically perturbing the sub-region of tissue 44, the permittivity of the tissue 44 can be altered relative to the applied electric field 36 to generate a displacement current 32 in addition to the current that would be present due to the source electric field 36 and altered due to conductivity changes in the tissue caused by the mechanical perturbation.

By providing the mechanical field 38 to the sub region of tissue 44, the permittivity can be altered within the electric field 36 by either new elements of the sub region of tissue 44 vibrating in and out of the electric field such that the continuum permittivity of the tissue is changed relative to the electric field 36, or that the bulk properties of the sub region of tissue 44 and the permittivity, or tissue capacitance, change due to the mechanical perturbation. An example of altering the permittivity within the electric field can occur when a cell membrane and extra-cellular fluid, both of different permittivities, are altered in position relative to the electric field by the mechanical field. This movement of tissues of different permittivity relative to the electric field will generate a new displacement current. The tissues could have permittivity values as high as or greater than 10̂8 times the permittivity of free space, differ by orders of magnitude, and/or have anisotropic properties such that the tissue itself demonstrates a different permittivity magnitude depending on the relative direction of the applied electric field. An example of altering permittivity of the bulk tissue occurs where the relative permittivity constant of the bulk tissue is directly altered by mechanical perturbation in the presence of an electric field. The mechanical source, i.e., ultrasound source may be placed at any location relative to the electrode locations, i.e., within or outside the same location as the electrodes, as long as components of the electric field and mechanical field are in the same region.

Tissue permittivities can be altered relative to the applied electric fields via a number of methods. Mechanical techniques can be used to either alter the bulk tissue permittivity relative to an applied electric field or move tissue components of differing permittivities relative to an applied electric field. There are no specific limitations to the frequency of the mechanical field that is applied as previously discussed, however, exemplary frequencies range from the sub kHZ to 1000s of MHz. A second electromagnetic field could be applied to the tissue, at a different frequency than the initial frequency of the applied electromagnetic field, such that it alters the tissue permittivity at the frequency dependent point of the initially applied electric field. An optical signal could also be focused on the tissues to alter the permittivity of the tissue relative to an applied electric field. A chemical agent or thermal field could also be applied to the tissues to alter the permittivity of the tissue relative to an applied electric field. These methods could also be used in combination to alter the tissue permittivity relative to an applied electric field via invasive or noninvasive methods.

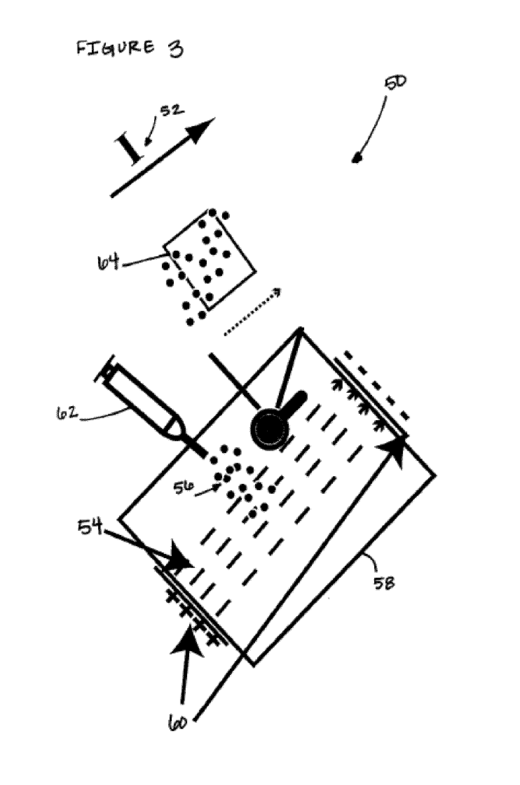

For example, FIG. 3 shows a set up 50 for generating an altered current with a newly generated displacement current 52 through the combined effects of an electric field 54 and a chemical agent 56. A tissue or composite of tissues 58 is placed within an electric source 60 which generates an electric field 54 and combined with chemical source 62 which releases a chemical agent 56 that can be focused on the tissue 58. In the area that the chemical agent 56 is released in the tissue 64, the electric field 54 transects the sub region of tissue 64, and the chemical agent 56 reacts with the sub region of tissue 64 to alter the tissue's relative permittivity relative to the applied electric field 54. This generates a displacement current 52 in addition to the current that would be present due to the source electric field 54. The chemical agent 56 may be any agent which can react with the tissue or cellular components of the tissue 64 to alter its permittivity relative to the electric field 54. This may be by a thermoreactive process to raise or lower the tissue 64 temperature or through a chemical reaction which alters the distribution of ions in the cellular and extra-cellular media, for instance, along ionic double layers at cell walls in the tissue 64. Similarly, the conformation of proteins and other charged components within the tissue 64 could be altered such that the permittivity of the tissue is altered relative to the low frequency electric field 54. The agent could also be any agent that adapts the permanent dipole moments of any molecules or compounds in the tissue 64, temporarily or permanently relative to the low frequency electric field 54. The chemical reaction driven by the chemical agent 56 must work rapidly enough such that the permittivity of the tissue is quickly altered in the presence of the electric field 54 in order to generate the displacement current 52. The reaction may also be such as to fluctuate the permittivity, such that as the permittivity continues to change displacement currents continue to be generated. In addition to the main permittivity change that occurs in the tissues, a conductivity change could also occur in the tissue, which secondarily alters the ohmic component of the currents. A biological agent may be used in place of, or in addition to, the chemical agent 56. This embodiment may have particular application for focused drug delivery where an additional chemical or biological agent is included to assist in therapy of the tissue, or where the altered current could drive an additional electrochemical reaction for therapy. For example, this could be used in areas such as focused gene therapy or focused chemotherapy.

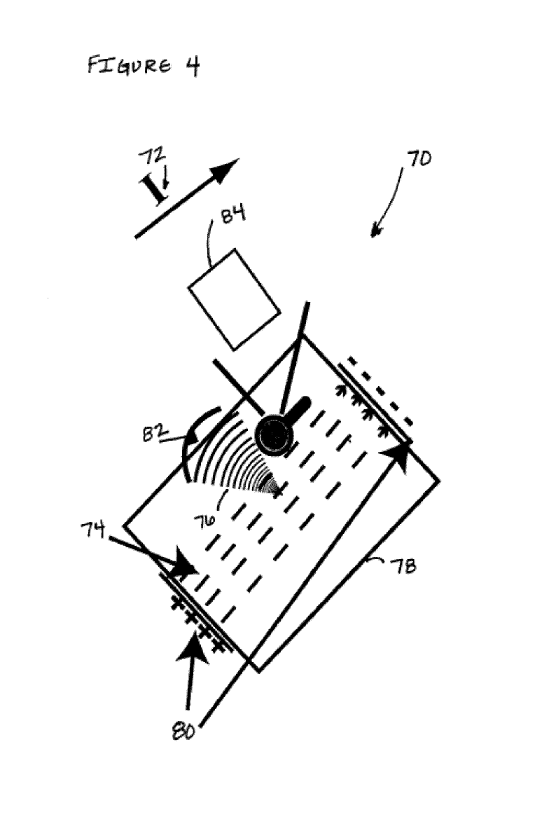

Another example is shown in FIG. 4, which illustrates a set up 70 for applying a method for generating an altered current with a newly generated displacement current 72 through the combined effects of a low frequency electric field 74 and an electromagnetic radiation field 76. A tissue or composite of tissues 78 is placed within a low frequency electric field 74 which is generated by an electric source 80 and combined with radiation source 82 which generates a radiation field 76 that can be focused on the tissue 78. In the area that the radiation field 76 is focused in the tissue 78, the electric field 74 transects the sub component of tissue 84, where the radiation field 76 interacts with the sub component of tissue 84 to alter the tissue's relative permittivity relative to the applied electric field 74, and as such generates a displacement current 72 in addition to the current that would be present due to the source electric field 74 or the radiation source field 76 alone. The electromagnetic radiation field 76 could, for example, interact with the tissue 84 by altering its temperature through ohmic processes, alter the distribution of ions in the cellular and extra-cellular media for instance along ionic double layers along cell walls through the electric forces acting on the ions, or alter the conformation of proteins and other charged components within the tissue through the electric forces such that the permittivity of the tissue is altered relative to the low frequency electric field 74. Furthermore, the electromagnetic field 76, could interact with the tissue 84 by moving components of the tissue via electrorestrictive forces, as would be seen in anisotropic tissues, to alter the continuum permittivity of the tissue relative to the low frequency electric field 74. In addition to the main permittivity change that occurs in the tissues, a conductivity change could also occur in the tissue, which secondarily alters the ohmic component of the currents.

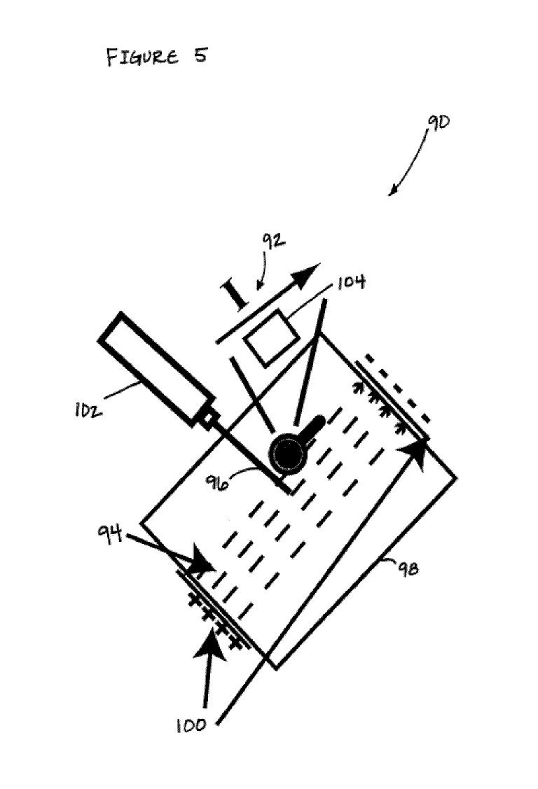

FIG. 5 shows a set up 90 for applying a method for generating an altered current with a newly generated displacement current 92 through the combined effects of an electric field 94 and an optical beam 96. A tissue or composite of tissues 98 is placed within electric field 94 generated by an electric source 100 and combined with optical source 102 which generates optical beam 96 that can be focused on the tissue 98. In the area that the optical beam 96 is focused on the tissue, the electric field 94 transects the sub component of tissue 104, where the optical beam 96 reacts with the tissue to alter the tissue's relative permittivity relative to the applied electric field 94, and as such generates a displacement current 92 in addition to the current that would be present due to the source electric field 94. The optical beam 96 could, for example, interact with the tissue by altering its temperature through photothermal effects and/or particle excitation, alter the distribution of ions in the cellular and extra-cellular media for instance along ionic double layers along cell walls by exciting the movement of ions optically, ionizing the tissue via laser tissue-interactions, or alter the conformation of proteins and other charged components within the tissue such that the permittivity of the tissue is altered relative to the low frequency electric field 94. In addition to the main permittivity change that occurs in the tissues, a conductivity change could also occur in the tissue, which secondarily alters the ohmic component of the currents.

In another embodiment, a thermal source to alter the permittivity of the tissue may be used. In such embodiments, a thermal source such as a heating probe, a cooling probe, or a hybrid probe may be placed external or internal to the tissue to be stimulated. A thermal source may alter the permittivity of the tissue through the direct permittivity dependence of tissue temperature, mechanical expansion of tissues in response to temperature changes, or by mechanical forces that arise due to altered particle and ionic agitation in response to the temperature alteration such that permittivity of the tissue is altered relative to an applied electric field. In addition to the main permittivity change that occurs in the tissues, a conductivity change could also occur in the tissue, which secondarily alters the ohmic component of the currents. This embodiment may be useful for stimulation in the presence of an acute injury to the tissue where the thermal source could be used to additionally assist in the treatment of the tissue injury, for example with a traumatic brain injury or an infarct in any organ such as the heart. The tissue could be cooled or heated at the same time stimulation is provided to reduce the impact of an injury.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein.

### EXAMPLES

### Example 1

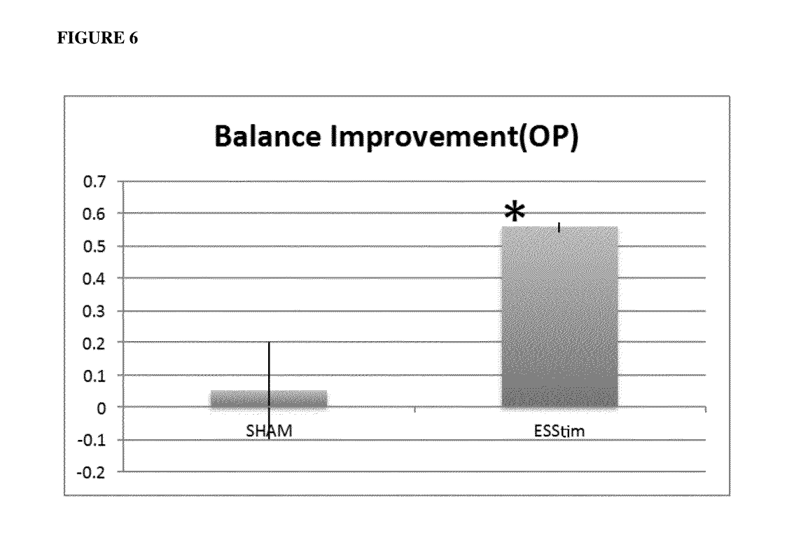

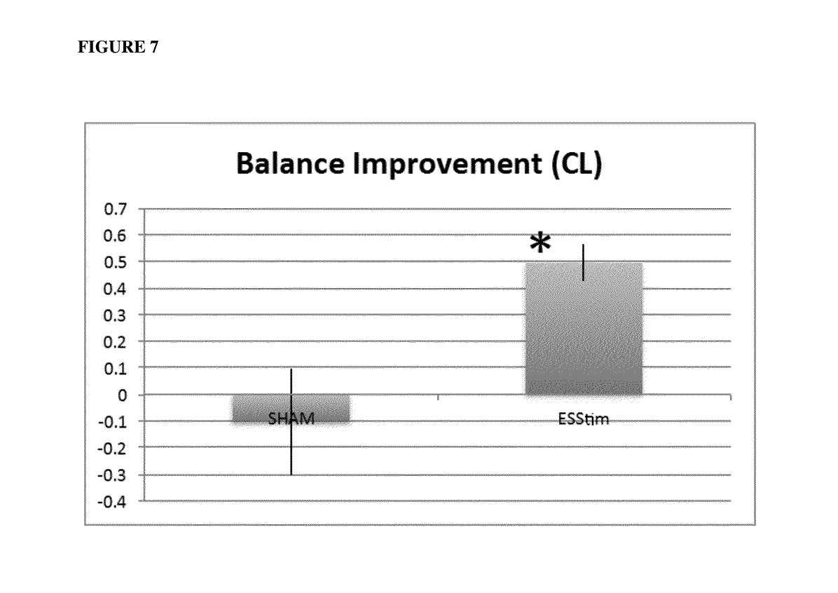

In a set of 4 Parkinson's disease (PD) patients (2 active Electrosonic Stimulation, ESStim), 2 SHAM), stimulation was provided above the patients left M1 for 2 weeks, 5 days/week, 20 minutes per day. Patient's balance was examined at baseline, prior to any stimulation, and at the end of the stimulation period. Patient's balance was assessed with both their eyes open and their eye's closed. The data in FIG. 6 show that the active stimulation group improved by 55% compared to baseline with their eyes open (OP). The SHAM stimulation group improved by 5% compared to baseline with their eyes open. The data in FIG. 7 show that the active stimulation group improved by 49% compared to baseline with their eye's closed (CL). The SHAM stimulation group decreased by 10% compared to baseline with their eye's closed. Both comparisons were significant (p<0.05) as analyzed via an unpaired t-test (p=0.02 for eyes open and p=0.03 for eye's closed).

### Example 2

We focused on evaluating Electrosonic Stimulation Effects on a walking ability/gait test in PD patients who received Electrosonic Stimulation provided over the primary motor cortex (M1) for 10 days, 20 minutes/day (12 Active, 12 SHAM). As proposed, all patients were provided stimulation and evaluated in the ‘On’ state. We have provided stimulation to patient completion in 20 patients at the time this report was generated, with 4 patients ongoing treatment and analysis. This efficacy data is focused on the 20 patients who have completed all of their stimulation sessions. We tested:
Walking Time/Gait: Times were measured for a patient to walk 10 m. This was done three times and averaged.
   We designed the study to compare Walking Time improvements between the Active

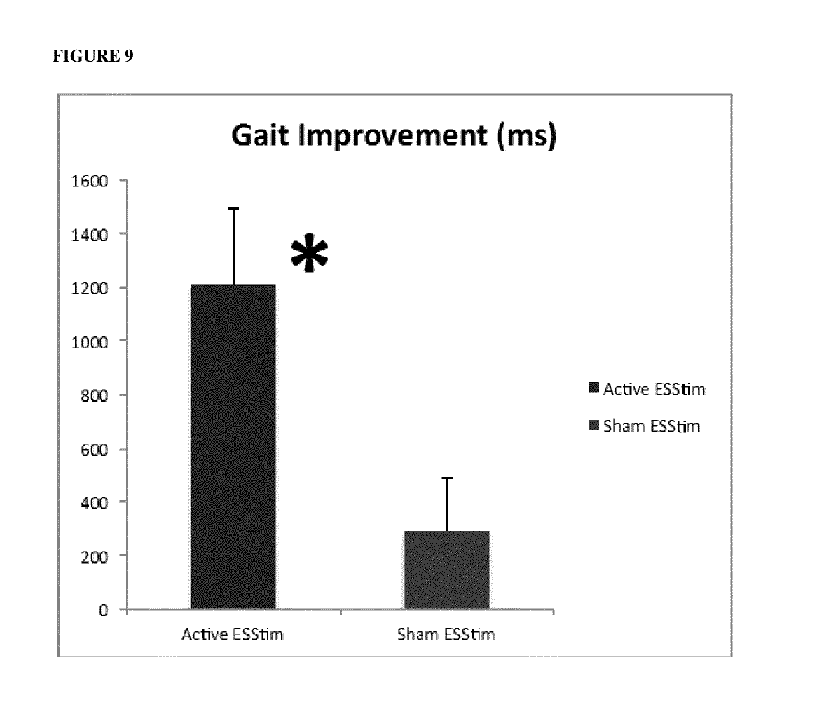

Electrosonic Stimulation and SHAM Electrosonic Stimulation conditions on the last day of stimulation (i.e. the difference in times to walk ten meters as measured at baseline and following the last day of stimulation (i.e., day 10)). We demonstrated a significant improvement in walking times comparing Active and SHAM stimulations, with 1212 ms vs 294 ms improvements following the last stimulation session compared to baseline (p=0.0063, t-test). This represents a 312.98% change relative to SHAM. See FIG. 9.

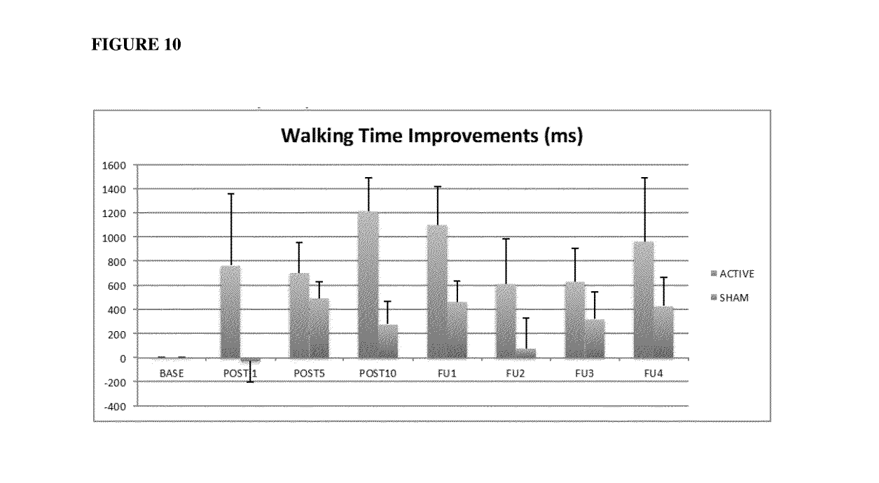

Given that we have acquired additional data from these patients (from measurements at the baseline (Base), following the first stimulation session (Post 1), following the 5 stimulation session (Post 5), following the 10th stimulation session (Post 10), at the first follow-up visit 1 week post stimulation (FU1), at the second follow-up visit 2 weeks post stimulation (FU2), at the third follow-up visit 1 month post stimulation (FU3), and at the last follow-up visit (FU2)) we analyzed the data via 2 way-ANOVA (Dependent: Improvement in Walking Time/Independent: Visit, Stimulation Type) and demonstrated a significant effect for Stimulation Type (p=0.0029). See FIG. 10.

Secondarily, we also performed a paired analysis where patient were paired based on their baseline UPDRS scores and ran a 3 way-ANOVA (Dependent: Improvement in Walking Time for Baseline Independent: Visit, Stimulation Type, and Patient Pairing (paired based on baseline performance)) and demonstrated a significant effect for Stimulation Type (p=0.01), Visit (p=0.024), and Pairing (p=0.003) and significant interaction effects for Pairing and Stimulation Type (p=0.004). This interaction effect between stimulation type and pairing suggests that Electrosonic Stimulation may be particularly effective, relative to sham stimulation, in a subpopulation of patients, based on their baseline scores before stimulation. Specifically, we found that patients with smaller baseline scores were more likely to demonstrate a large improvement in walking time with Electrosonic Stimulation, relative to sham stimulation.

What is claimed is:

1. A method for improving balance of a subject, the method comprising: noninvasively providing stimulation to a central nervous system of an awake subject to modulate a signal sent to or from the awake subject's central nervous system, wherein the stimulation to the central nervous system improves balance of the subject.

2. The method according to claim 1, wherein the method is performed without stimulating the peripheral nervous system.

3. The method according to claim 1, wherein the stimulation is provided in a single session that lasts three hours or less.

4. The method according to claim 1, wherein the method is performed in combination with use of a therapeutic agent.

5. The method according to claim 1, wherein the subject is afflicted with a movement disorder.

6. The method according to claim 5, wherein the movement disorder is selected from the group consisting of: Parkinson's disease; Parkinsonism; Dystonia; Cerebral Palsy; Bradykinesia; Chorea; Huntington's Disease; Ataxia; Tremor; Essential Tremor; Myoclonus; tics; Tourette Syndrome; Restless Leg Syndrome; and Stiff Person Syndrome.

7. The method according to claim 1, wherein the stimulation is provided by a combination of an electric field and a mechanical field.

8. The method according to claim 7, wherein the mechanical field is generated by an ultrasound device.

9. The method according to claim 7, wherein said electric field is pulsed.

10. The method according to claim 7, wherein the electric field is time varying.

11. The method according to claim 7, wherein the electric field is pulsed a plurality of times, and each pulse may be for a different length of time.

12. The method according to claim 7, wherein said electric field is time invariant.

13. The method according to claim 7, wherein the mechanical field is pulsed.

14. The method according to claim 7, wherein the mechanical field is time varying.

15. The method according to claim 7, wherein the mechanical field is pulsed a plurality of times, and each pulse may be for a different length of time.

16. The method according to claim 7, wherein the stimulation is applied to more than one structure within the brain.

17. The method according to claim 7, wherein the electric field is a DC electric field.

18. The method according to claim 1, wherein the stimulation of the central nervous system is provided to the brain.

19. The method according to claim 7, wherein the combined effect of the electric field and the mechanical field alters neural function past the duration of stimulation.
